(19) United States
(12) Patent Application Publication
(10) Pub. No.: US 2024/0207644 A1
(43) Pub. Date: Jun. 27, 2024

(54) RADIATION DOSE PREDICTION VIA ARTIFICIAL INTELLIGENCE MODELS USING ORGAN DOSE TRADE-OFF

(71) Applicant: Siemens Healthineers International AG, Steinhausen (CH)

(72) Inventors: Simeng Zhu, Troy, MI (US); Maria Isabel Cordero Marcos, Espoo (FI); Elena Czeizler, Helsinki (FI); Supratik Bose, San Ramon, CA (US); Anthony Magliari, Newark, IL (US)

(73) Assignee: Siemens Healthineers International AG, Steinhausen (CH)

(21) Appl. No.: 18/086,374

(22) Filed: Dec. 21, 2022

(51) Int. Cl. A61N 5/10; G06N 3/08; G16H 20/40

(52) U.S. Cl. CPC A61N 5/1031; A61N 5/1048; G06N 3/08; G16H 20/40; A61N 2005/1074

(57) ABSTRACT

Provided herein are methods and systems to train and execute a model that uses artificial intelligence methodologies to learn and predict dosages administrated to different structures during radiotherapy treatment. A method comprises receiving a value indicating a prioritization between a first organ at risk of a patient and a second organ at risk of the patient receiving radiation dosage; executing an artificial intelligence model using the value to predict a radiation dosage for the first organ at risk, the second organ at risk, and a target structure, wherein the artificial intelligence model is trained in accordance with a training dataset comprising a set of participants, dosage administered to the participant's target structure, first organ at risk, and second organ at risk; and outputting the predicted radiation dosage for at least one of the first organ at risk, the second organ at risk, and a target structure.

### TECHNICAL FIELD

This application relates generally to using machine-learning techniques to train artificial intelligence models configured to predict dosage distribution attributes for radiotherapy treatment.

### BACKGROUND

In the treatment planning of radiotherapy, the prediction of the expected radiation dose distribution to be delivered is an important task that can improve the efficiency of treatment planning. Specifically, dose prediction can be used to effectively drive an optimizer computer model (also referred to herein as the plan optimizer). For instance, the optimizer may ingest the dose predictions and generate a plan accordingly. Moreover, the dose prediction can be used to serve as a clinical decision support tool prior to running the optimizer.

Currently, many software solutions use algorithmic methods to calculate a predicted dose distribution for a patient structure, such as a planning target volume (PTV) or an organ at risk (OAR). For instance, many software solutions use computer models that utilize artificial intelligence (AI) to predict the dosage that could or would be delivered to a structure (e.g., anatomical structure or a patient organ). However, these conventional software solutions suffer from technical challenges. For instance, most current deep learning approaches are only able to predict one dose distribution instance for a given set of planning structures, corresponding to a specific treatment intent (e.g., a specific trade-off pattern). Therefore, current software solutions are incapable of simultaneously producing several dose distributions that consider alternative trade-off patterns between planning structures.

### SUMMARY

For the aforementioned reasons, there is a desire for a system that can train one or more AI models that enable the prediction of radiation dose distributions with various trade-off patterns, as requested by an end-user (e.g., a clinician or a treating physician). Using the methods and systems discussed herein, a clinician can select the desired trade-off levels between different structures, which will subsequently be used to produce a three-dimensional (3D) radiation dose distribution reflecting the selected trade-off patterns.

The methods and systems described herein utilize a specially designed AI model trained using machine-learning technique that is data-driven, whereas other solutions are often based on real-time calculation of multi-criteria optimization. Using the methods and systems described herein, dose predictions can be made much faster than other methods. For instance, a dose can be predicted quickly (e.g., in a few seconds), while other data-driven techniques can require a few minutes for calculation. Therefore, using the methods and systems discussed herein is more efficient than other methods. Specifically, the AI model disclosed herein is used as the input to a plan optimizer, whereas other solutions are used after a plan has been generated via the plan optimizer.

Other solutions utilize known algorithms (e.g., mixture density network algorithms), but experience some technical shortcomings. For instance, those solutions can only predict a limited, sometimes predefined, number of radiation dose distributions per patient, whereas the AI model disclosed herein allows the prediction of a large number of doses, including also trade-off patterns not illustrated in the training set.

Other solutions use U-Net (a convolutional neural network) in an attempt to achieve the same results. However, those solutions require a prohibitively high number of training data and large training datasets. In contrast, the AI model disclosed herein may use as few as three plans for each patient in the training set to generate acceptable results, because the AI model disclosed herein may use a generative model specialized for this task. In some embodiments, the generative model may use conditional variational encoders to train the model. However, the methods and systems discussed herein are not limited to generative models and/or variational encoders (or conditional variational encoders).

In an embodiment, a method comprises receiving, by a processor, a value indicating a prioritization between a first organ at risk of a patient and a second organ at risk of the patient receiving radiation dosage; executing, by the processor, an artificial intelligence model using the value to predict a radiation dosage for the first organ at risk, the second organ at risk, and a target structure, wherein the artificial intelligence model is trained in accordance with a training dataset comprising a set of participants, dosage administered to the participant's target structure, first organ at risk, and second organ at risk; and outputting, by the processor, the predicted radiation dosage for at least one of the first organ at risk, the second organ at risk, or a target structure.

Outputting the predicted dosage may comprise displaying a dose-volume histogram depicting the predicted radiation dosage for at least one of the first organ at risk, the second organ at risk, or a target structure.

The value indicating the prioritization may be received via a sliding scale input element.

The processor may receive a plurality of values indicating a plurality of prioritizations between the first organ at risk of the patient and the second organ at risk of the patient receiving radiation dosage and outputs a plurality of predicted radiation dosages.

The method may further comprise transmitting, by the processor, the predicted radiation dosage to a plan optimizer software solution.

The method may further comprise adjusting, by the processor, at least one attribute of a radiotherapy machine in accordance with the predicted radiation dosage.

The artificial intelligence model may be trained using a set of weighted tensors corresponding to the training dataset.

The artificial intelligence model may be trained using a generative artificial intelligence model corresponding to variational auto-encoder or a conditional variational auto-encoder.

In another embodiment, a system comprises a server comprising a processor and a non-transitory computer-readable medium containing instructions that when executed by the processor causes the processor to receive a value indicating a prioritization between a first organ at risk of a patient and a second organ at risk of the patient receiving radiation dosage; execute an artificial intelligence model using the value to predict a radiation dosage for the first organ at risk, the second organ at risk, and a target structure, wherein the artificial intelligence model is trained in accordance with a training dataset comprising a set of participants, dosage administered to the participant's target structure, first organ at risk, and second organ at risk; and output the predicted radiation dosage for at least one of the first organ at risk, the second organ at risk, or a target structure.

Outputting the predicted dosage may comprise displaying a dose-volume histogram depicting the predicted radiation dosage for at least one of the first organ at risk, the second organ at risk, or a target structure.

The value indicating the prioritization between the first organ at risk of the patient and the second organ at risk of the patient may be received via a sliding scale input element.

The processor may receive a plurality of values indicating a plurality of prioritizations between the first organ at risk of the patient and the second organ at risk of the patient receiving radiation dosage and outputs a plurality of predicted radiation dosages.

The instructions may further cause the processor to transmit the predicted radiation dosage to a plan optimizer software solution.

The instructions may further cause the processor to adjust at least one attribute of a radiotherapy machine in accordance with the predicted radiation dosage.

The artificial intelligence model may be trained using a set of weighted tensors corresponding to the training dataset.

The artificial intelligence model may be trained using a generative artificial intelligence model corresponding to a variational auto-encoder or a conditional variational auto-encoder.

In another embodiment, a system comprises a computer in communication with a server and configured to display a graphical user interface; a radiotherapy machine in communication with the server; and the server configured to receive a value indicating a prioritization between a first organ at risk of a patient and a second organ at risk of the patient receiving radiation dosage; execute an artificial intelligence model using the value to predict a radiation dosage for the first organ at risk, the second organ at risk, and a target structure, wherein the artificial intelligence model is trained in accordance with a training dataset comprising a set of participants, dosage administered to the participant's target structure, first organ at risk, and second organ at risk; and output the predicted radiation dosage for at least one of the first organ at risk, the second organ at risk, or a target structure.

Outputting the predicted dosage may comprise displaying a dose-volume histogram depicting the predicted radiation dosage for at least one of the first organ at risk, the second organ at risk, or a target structure.

The value indicating the prioritization may be received via a sliding scale input element.

The server may receive a plurality of values indicating a plurality of prioritizations between the first organ at risk of the patient and the second organ at risk of the patient receiving radiation dosage and outputs a plurality of predicted radiation dosages.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates components of an artificial intelligence (AI) dosage modeling system, according to an embodiment.

FIG. 2 illustrates a process flow diagram of AI dosage modeling system, according to an embodiment.

FIG. 3 illustrates a process flow diagram of AI dosage modeling system, according to an embodiment.

FIG. 4 illustrates a process flow diagram of AI dosage modeling system, according to an embodiment.

FIG. 5 illustrates a process a flow diagram for generating a training dataset, according to an embodiment.

FIG. 6 illustrates a process a flow diagram for generating a training dataset, according to an embodiment.

FIG. 7 illustrates a process a flow diagram for generating a training dataset, according to an embodiment.

FIG. 8 illustrates a process a flow diagram for training an AI model, according to an embodiment.

FIG. 9 illustrates a process a flow diagram for executing an AI model, according to an embodiment.

FIGS. 10-11 illustrate different process flow diagrams and examples of training and execution of an AI dosage modeling system, according to an embodiment.

FIG. 12 illustrates different dosages predicted in an AI dosage modeling system, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

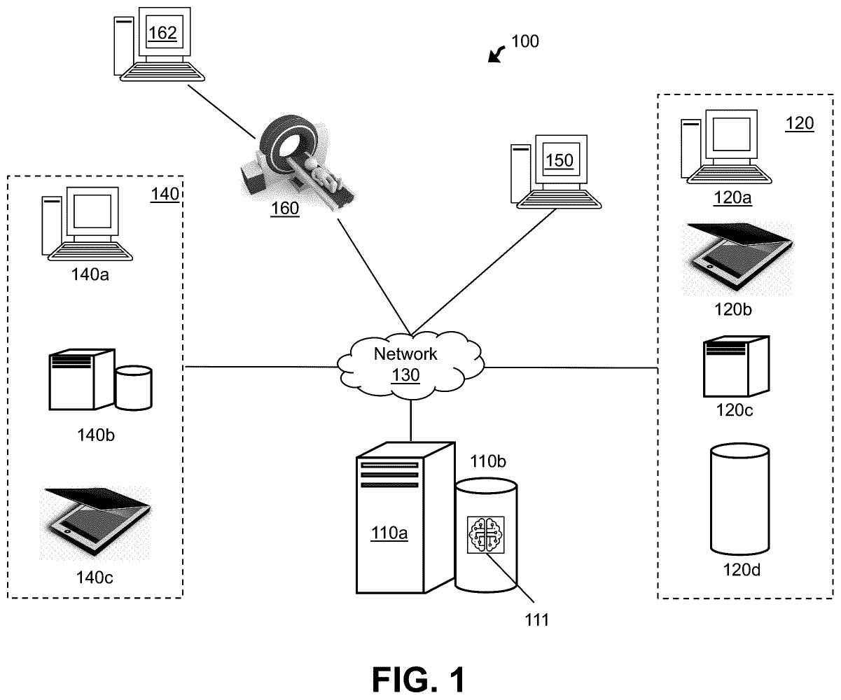

FIG. 1 illustrates components of a system 100 for an AI dose modeling system, according to an embodiment. The system 100 may include an analytics server 110a, system database 110b, an AI model 111, electronic data sources 120a-d (collectively electronic data sources 120), end-user devices 140a-c (collectively end-user devices 140), an administrator computing device 150, a medical device 160, and a medical device computer(s) 162. Various components depicted in FIG. 1 may belong to a radiotherapy clinic at which patients may receive radiotherapy treatment, in some cases via one or more radiotherapy machines (e.g., medical device 160) located within the clinic.

The system 100 is not confined to the components described herein and may include additional or other components not shown for brevity, which are to be considered within the scope of the embodiments described herein.

The above-mentioned components may be connected to each other through a network 130. Examples of the network 130 may include, but are not limited to, private or public local-area-networks (LAN), wireless LAN (WLAN) networks, metropolitan area networks (MAN), wide-area networks (WAN), and the Internet. The network 130 may include wired and/or wireless communications according to one or more standards and/or via one or more transport media. Communication over the network 130 may be performed in accordance with various communication protocols such as Transmission Control Protocol and Internet Protocol (TCP/IP), User Datagram Protocol (UDP), and Institute of Electrical and Electronics Engineers (IEEE) communication protocols. In one example, the network 130 may include wireless communications according to Bluetooth specification sets or another standard or proprietary wireless communication protocol. In another example, the network 130 may also include communications over a cellular network, including, e.g., GSM (Global System for Mobile Communications), CDMA (Code Division Multiple Access), or EDGE (Enhanced Data for Global Evolution) networks.

The analytics server 110a may generate and display an electronic platform configured to receive patient information and output results of execution of the AI model 111. The electronic platform may include a graphical user interface (GUI) displayed on the electronic data sources 120, the end-user devices 140, the medical device 160, and/or the administrator computing device 150. An example of the electronic platform generated and hosted by the analytics server 110a may be a web-based application or a website configured to be displayed on various electronic devices, such as mobile devices, tablets, personal computers, and the like.

The analytics server 110a may be any computing device comprising a processor and non-transitory, machine-readable storage capable of executing the various tasks and processes described herein. The analytics server 110a may employ various processors such as a central processing unit (CPU) and graphics processing unit (GPU), among others. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

The electronic data sources 120 may represent various sources that contain, retrieve, and/or access data associated with a medical device 160, such as operational information associated with previously performed radiotherapy treatments (e.g., electronic log files or electronic configuration files), data associated with previously monitored patients (e.g., tumor location and calculated dosage distributions), or participants in a studies for training the AI models discussed herein. For instance, the analytics server 110a may use the clinic computer 120a, medical professional device 120b, server 120c (associated with a treating physician and/or clinic), and database 120d (associated with the physician and/or the clinic) to retrieve and receive data. The analytics server 110a may also retrieve the data from the electronic data sources 120, generate a training dataset, and train the AI model 111, accordingly. The analytics server 110a may execute various algorithms to translate raw data received or retrieved from the electronic data sources 120 into machine-readable objects that can be stored and processed by other analytical processes as described herein.

End-user devices 140 may be any computing device comprising a processor and a non-transitory, machine-readable storage medium capable of performing the various tasks and processes described herein. Non-limiting examples of an end-user device 140 may be a workstation computer, laptop computer, tablet computer, and server computer. During operation, various users may use end-user devices 140 to access the GUI operationally managed by the analytics server 110a. Specifically, the end-user devices 140 may include clinic computer 140a, clinic server 140b, and a medical processional device 140c. Even though referred to herein as “end-user” devices, these devices may not always be operated by end-users. For instance, the clinic server 140b may not be directly used by an end-user. However, the results stored onto the clinic server 140b may be used to populate various GUIs accessed by an end-user via the medical professional device 140c.

The administrator computing device 150 may represent a computing device operated by a system administrator. The administrator computing device 150 may be configured to display radiotherapy treatment attributes generated by the analytics server 110a (e.g., various analytic metrics determined during training of one or more machine learning models and/or systems); monitor various models 111 utilized by the analytics server 110a, electronic data sources 120, and/or end-user devices 140; review feedback; and/or facilitate training or retraining (calibration) of the AI model 111 that are maintained by the analytics server 110a.

The medical device 160 may be a radiotherapy machine configured to implement a patient's radiotherapy treatment. The medical device 160 may also include an imaging device capable of emitting radiation, such that the medical device 160 may perform imaging according to various methods to accurately image the internal anatomical structure of a patient. For instance, the medical device 160 may include a rotating system (e.g., a static or rotating multi-view system). A non-limiting example of a multi-view system may include stereo systems (e.g., two systems arranged orthogonally). The medical device 160 may also be in communication with a medical device computer 162 that is configured to display various GUIs discussed herein. For instance, the analytics server 110a may display the results predicted by the AI model 111 onto the computing devices described herein.

The AI model 111 may be stored in the system database 110b. The AI model 111 may be trained using data received or retrieved from the electronic data sources 120 and may be executed using data received from the end-user devices, the medical device 160, and/or other sensors. In some embodiments, the AI model 111 may reside within a data repository that is local or specific to a clinic. In various embodiments, the AI model 111 uses one or more engines (e.g., deep learning engines) to generate a predicted dose for one or more anatomical structures of a patient. The predicted dosage may then be used in a down-stream software application or a computer model, such as a plan optimizer.

It should be understood that any alternative and/or additional machine learning model(s) may be used to implement similar learning engines. The deep learning engines can include processing pathways that are trained during a training phase. Once trained, deep learning engines may be executed (e.g., by the analytics server 110a) to generate predicted results.

As described herein, the analytics server 110a may store the AI model 111 (e.g., neural networks, random forest, support vector machines, regression models, recurrent models, etc.) in an accessible data repository. The analytics server 110a may retrieve the AI model 111 and train it to predict a deformity associated with one or more of the patient's structures and/or organs. Various machine-learning techniques may involve training the machine learning models to predict a dose for one or more structures, including supervised learning techniques, unsupervised learning techniques, or semi-supervised learning techniques, among others.

One type of deep learning engine is a deep neural network (DNN). A DNN is a branch of neural networks and consists of a stack of layers each performing a specific operation (e.g., convolution, pooling, loss calculation, etc.) Each intermediate layer receives the output of the previous layer as its input. The beginning layer is an input layer, which is directly connected to or receives an input data structure that includes the data items in one or more machine-readable objects, and may have a number of neurons equal to the data items in one or more machine-readable objects provided as input. For example, a machine-readable object may be a data structure, such as a list or vector, which includes a number of data fields containing data received in order to predict a dosage. Each neuron in an input layer can accept the contents of one data field as input. The analytics server 110a may pre-process the machine-readable objects (e.g., through an encoding process) such that the data fields may be accepted as input to the AI model 111 described herein.

A next set of layers can include any type of layer that may be present in a DNN, such as a convolutional layer, a fully connected layer, a pooling layer, or an activation layer, among others. Some layers, such as convolutional neural network layers, may include one or more filters. The filters, commonly known as kernels, are of arbitrary sizes defined by designers. Each neuron can respond only to a specific area of the previous layer, called receptive field. The output of each convolution layer can be considered as an activation map, which highlights the effect of applying a specific filter on the input. Convolutional layers may be followed by activation layers to apply non-linearity to the outputs of each layer. The next layer can be a pooling layer that helps to reduce the dimensionality of the convolution's output. In various implementations, high-level abstractions are extracted by fully connected layers. The weights of neural connections and the kernels may be continuously optimized in the training phase.

The AI model may also be (but it does not have to be) a generative AI model. As used herein, generative AI models may refer to deep neural network models that attempt to learn an underlying probability distribution of its high dimensional training data by learning the probability distribution on a low dimensional (latent space) representation. At a later phase, e.g., during the “generation” mode of such model, it can sample the learned probability distribution on a low dimensional (latent space) representation of the data and generate high dimensional data that are similar to the data within the training dataset.

In a non-limiting example, a conditional generative neural network model can learn the probability distribution of hand written digits where it takes the “label” of the digit (“one”, “two” etc.) as condition and later generates realistic looking hand-written numbers given any such label.

The AI model 111 may use generative NN model(s) to learn the probability distribution of radiotherapy dose conditional on both organ and tumor segmentations as well as dose sparing ratios on such organs. While aspects of implantation of the AI model 111 is described as using conditional variational autoencoder (CVAE) as the chosen NN model, the methods and systems discussed herein are equally applicable to other generative models (e.g., Generative Adversarial Network (GAN)) when provided with same input as in CVAE.

In practice, training data may be user-generated through observations and experience, and used to facilitate learning. For example, training data may be received and monitored during past radiotherapy treatments provided to prior patients. In another example, the training data may be a dataset that includes dosage distribution of patients' PTVs while being treated and dose distribution of OARs for the same patients. Training data may be pre-processed via any suitable data augmentation approach (e.g., normalization, encoding, combinations thereof, etc.) to produce a new dataset with modified properties for improving model training. The methods and systems described herein are not limited to training AI models based on patients who have been previously treated. For instance, instead of previously treated patients, the training dataset may include data associated with any set of participants (not patients) who are willing to be monitored for the purposes of generating the training dataset. Therefore, participants in a study who are not being treated can be connected to one or more electronic sensors where the analytics server110a includes data collected from the sensors within the training dataset.

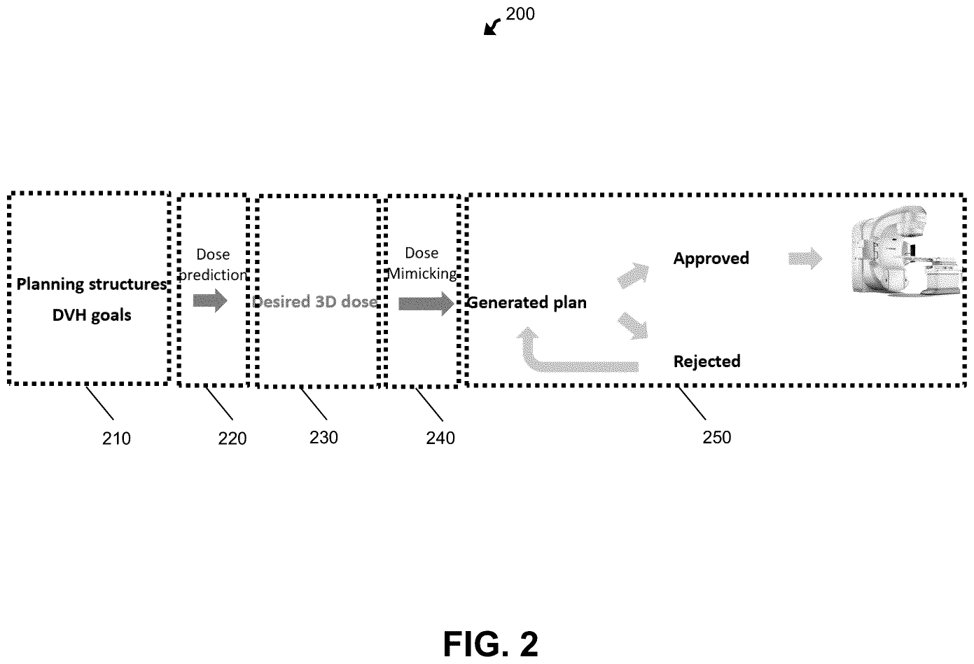

As illustrated in FIG. 2, dose prediction and treatment planning has traditionally been performed in a manner that does not account for different tradeoffs (e.g., the doses may only consider the average tradeoff inherent to the dataset that is used to train the dose prediction algorithm, but do not consider the endless other potential tradeoffs). For instance, the method 200 illustrates different steps involved in generating a treatment plan. A clinician may first identify clinical goals including planning structures and dose volume histogram (DVH) goals (step 210). After receiving the limits and parameters from the clinician, a computer model is typically used to generate dose predictions (step 220). The predicted doses are typically individualized and do not consider any tradeoffs (e.g., may only consider the average tradeoffs). For instance, the predicted dose may correspond to three organs considered individually. The predicted dosage will then drive the optimization and plan generation process. For instance, based on the predicted dosage, a 3D dosage can be generated (230), dose mimicking can be achieved (240), and a treatment plan can be generated (250). The treatment plan can then be presented to the clinician and the clinician can approve or reject the treatment plan.

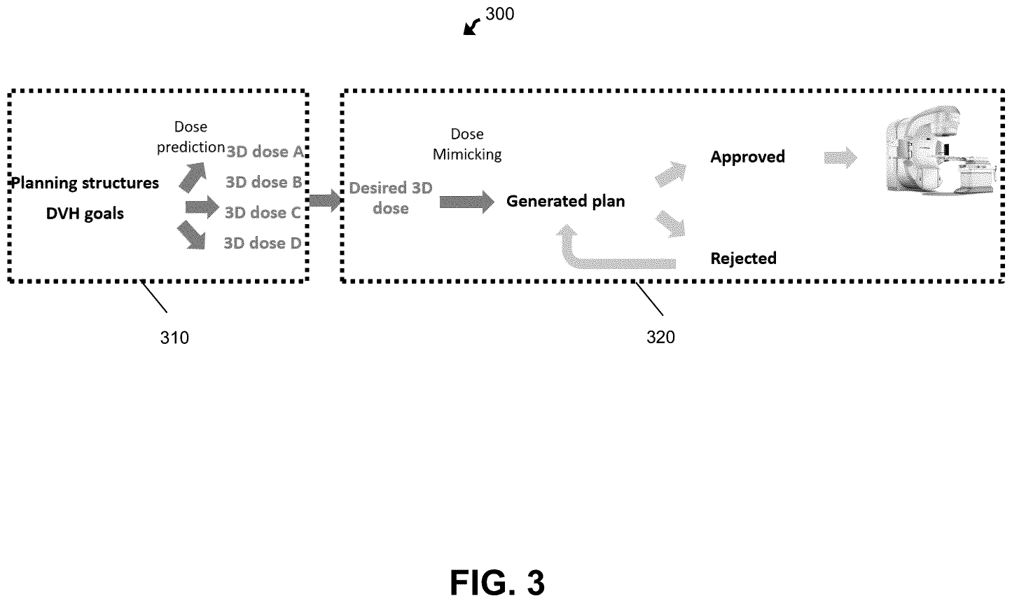

The methods and systems discussed herein use a different and/or additional paradigm in conjunction with different computer modeling techniques than those used previously. For instance, referring now to FIG. 3, a method 300 may use a computer model to predict multiple doses that consider tradeoffs between different structures (step 310), such that the clinician can select a combination of doses to be used for planning purposes (steps 320).

Using the methods and systems discussed herein, a software application may ingest the data produced by the disclosed AI model (where the calculations are customized for a particular patient's needs) to generate a customized treatment plan. For instance, a physician treating a patient suffering from prostate cancer may customize the dosage received by the prostate and its respective OARs (the rectum and bladder). Conventional systems may predict a 3D dose distribution for the prostate that is a standard treatment plan considering average tradeoffs (between different organs and structures) within the training data, which are not unique to each patient and produces generic results. However, a physician may determine that a particular patient may require shifting towards the bladder the dosage for the rectum, or vice versa. Therefore, conventional generic treatment plans are not desirable.

In contrast, using input received from a clinician (treating physician), the AI model discussed herein can generate customized dose distribution predictions. As a result, a downstream application, such as a plan optimizer, may ingest the data predicted by the computer model discussed herein to generate a treatment plan that is customized for a particular patient and based on prioritizations desired by the clinician.

In some embodiments, the clinician may receive multiple predictions for a series of organs and/or structures where each prediction corresponds to a different tradeoff. Therefore, the clinician may then determine which prediction is best suited for the patient. Accordingly, using the methods and systems discussed herein, the clinician may utilize the AI model to make a more informed decision that is customized for each patient. For instance, depending on patients' co-morbidities, the clinician may choose different tradeoffs for different patients.

Using the methods and systems discussed herein, the clinician may be provided different options to generate a treatment plan. For instance, given that the PTV (in this example, a prostate) is adequately covered (receives a dosage within the clinical objectives) and that the OAR constraints are met, there is a high degree of freedom to balance bladder vs. rectum doses (OAR constraints from clinical trial protocols tend to be loose). Accordingly, the AI model may still perform the tradeoff decisions within the constraints set by a clinician. The clinical dosage constraints may limit the number of possible permutations and tradeoffs while predicting dosages. However, it may not eliminate the possibility of generating multiple dosage predictions. Therefore, the dosage predictions discussed herein may not deviate from the objectives received from the clinician. Accordingly, using the methods and systems discussed herein allows the dosage for OARs to be customized for the patient while still remaining within the identified limits and clinical objectives.

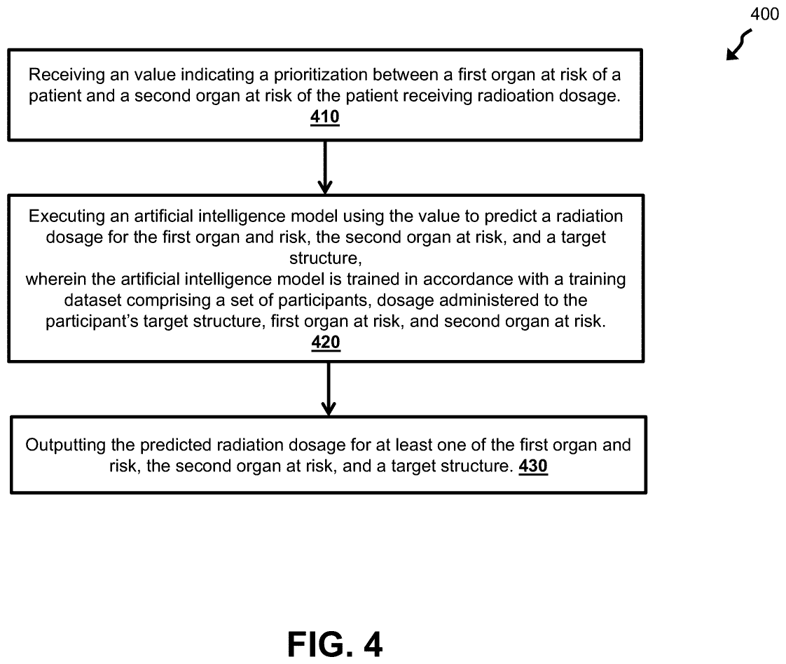

Referring to FIG. 4, depicted is an example data flow diagram 400 that shows how an AI model can be trained and executed to predict dosages, in accordance with an embodiment. The method 400 may include steps 410-430. However, other embodiments may include additional or alternative steps or may omit one or more steps altogether. The method 400 is described as being executed by a server, such as the analytics server described in FIG. 1. However, one or more steps of method 400 may be executed by any number of computing devices operating in the distributed computing system described in FIG. 1. For instance, one or more computing devices may locally perform part or all of the steps described in FIG. 4.

Using the method 400, an AI model can be trained in accordance with a training dataset to predict and/or generate 3D radiation dose distributions that are conditioned on patient anatomy and trade-off patterns between two (or more) planning structures (e.g., a PTV and one or more OARs). At implementation, using the method 400, the AI model may be executed to predict dose distribution in accordance with a clinicians requested prioritization values.

### Training of the AI Model

Before executing the AI model, the analytics server may first train the AI model and ensure its accuracy. The analytics server may train the AI model using a training dataset comprising data associated with a cohort of patients (or participants in a clinical trial).

The data required for model training may comprise 3D radiation dose volumes (three or more per patient case) and weighted anatomy tensors (three or more per patient case). Even though aspects of the examples used to describe training the AI model use three iterations per patient case, it is expressly understood that some other embodiments may use more or fewer iterations. Therefore, describing three different dose volumes per patient is not intended to limit the methods or systems discussed herein. Also, various examples discussed herein, describe Structure A and Structure B as two examples of OARs. However, it is expressly understood that the number of OARs can be more or fewer.

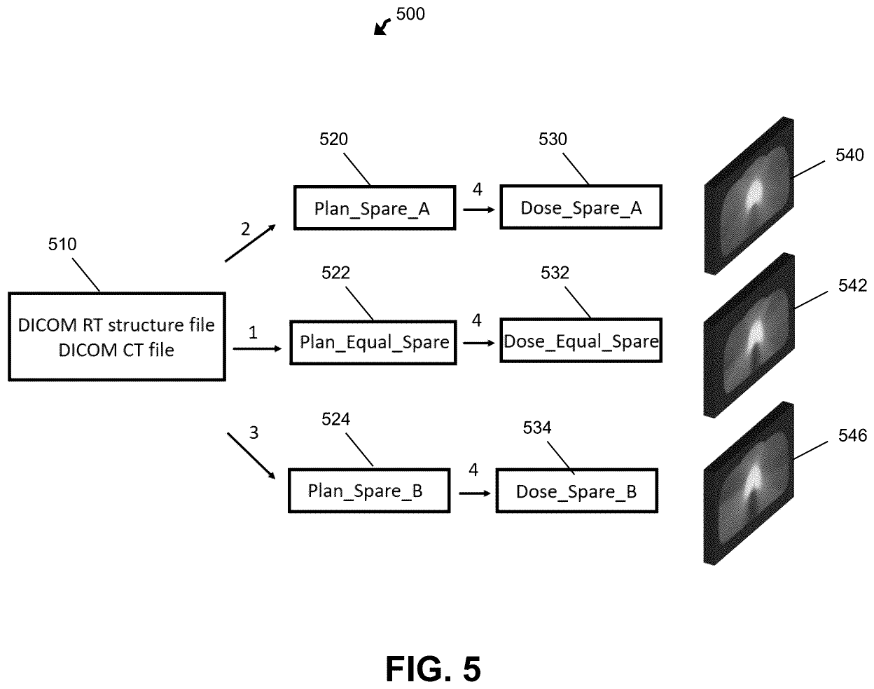

Referring now to FIG. 5, a method 500 illustrates a process a flow diagram for generating a training dataset, according to an embodiment. Specifically, the method 500 illustrates how the analytics server can generate a training dataset that can be used to train an AI model, such that the model is configured to generate prioritized dose distributions.

The analytics server may query and retrieve a set of previously performed dose predictions. For instance, the analytics server may retrieve a set of previously performed treatments and their corresponding treatment plans. In a non-limiting example, the analytics server may retrieve digital image communication in medicine (DICOM) radiotherapy CT files and/or a DICOM RT structure file associated with previously performed treatments or plan generations. In some embodiments, the information needed to generate the DICOM files may be created using participants in observed situations (e.g., where the participants are not being treated, but their information is being recorded for training purposes). DICOM, as used herein, may refer to a standard method for storing and transmitting digital medical image information, in which a file specially addresses the transmission of radiation therapy image data and/or the ancillary data.

The DICOM RT Structure file and DICOM CT file 510 may include all planning structures, including calculation data regarding the PTV and two or more OARs (e.g., Structure A and Structure B, the two structures of interest in terms of a trade-off decision). The DICOM RT Structure file and DICOM CT file 510 may also include one or more medical images associated with each patient/participant.

The analytics server may generate three iterations per patient case (e.g., each patient case may be represented by a DICOM RT structure file and a DICOM CT file). For instance, the analytics server may generate different iterations using a plan optimizer software solution (referred to also as the radiation plan optimizer). The plan optimizer may be a computer model that ingests DICOM files and generates a treatment plan (e.g., dose distribution).

Using a radiation plan optimizer (e.g., Photon Optimizer), the analytics server may generate a first radiation plan (Plan Equal Spare 522) in which Structures A and B are supposed to be spared equally. That is, structures A and B are equally treated as OARs when receiving inadvertent radiation. To achieve this, the analytics server may assign the value, p, as the priority value for terms in the objective function, which correspond to both Structures A and B.

Using the radiation plan optimizer, the analytics server may generate a second radiation plan called (Plan Spare A 520) in which the radiation dose for Structure A is supposed to be lower than that for Structure B (Structure A is prioritized over Structure B). Specifically, the Structure A may receive less dosage than it would in the Plan Equal Spare 522 while the dosage administered to the Structure B may be higher than the dosage of the Structure B in the Plan Equal Spare 522. To achieve this, the analytics server may use the same set of objectives and priority values as the ones used in generating the Plan Equal Spare 522. The analytics server may revise the priority values for terms in the objective function corresponding to Structures A and B to indicate that Structure A is prioritized over Structure B (e.g., 2p and p−20, respectively). In calculating the Plan Spare A 520, a higher the priority value for Structure A relative to Structure B may cause the radiation plan optimizer to reduce the dose allotted to Structure A.

The analytics server may also use the radiation plan optimizer to generate a third radiation plan (Plan Spare B 524) in which the radiation dose for Structure B is supposed to be lower than that for Structure A (Structure B is prioritized over Structure A). Specifically, the Structure B may receive less dosage than it would in the Plan Equal Spare 522 while the dosage administered to the Structure A may be higher than the dosage of the Structure A in the Plan Equal Spare 522. To achieve this, the analytics server may use the same set of objectives and priority values as the ones used in the other plans (520 and 522). The analytics server may revise the priority values for items in the objective function corresponding to Structures A and B to indicate that Structure A is prioritized over Structure B (e.g., p−20 and 2p, respectively). In calculating Plan Space B 524, a higher priority value for Structure B relative to Structure A may cause the radiation plan optimizer to reduce the dose allotted to Structure B.

The analytics server may generate multiple iterations for each patient case, such that each patient case has the following three plans:
A first plan that does not prioritize an OAR over another OAR;
A second plan that prioritizes a first OAR over the second OAR; and
A third plan that prioritize the second OAR over the first OAR.

After generating the plan, the analytics server may generate weighted anatomy tensors corresponding to the generated plans. A tensor, as used herein, may refer to any multi-dimensional array of data (e.g., vectors and/or matrices). Before generating the tensors, the analytics server may first separate medical images (corresponding to different plans) into component images.

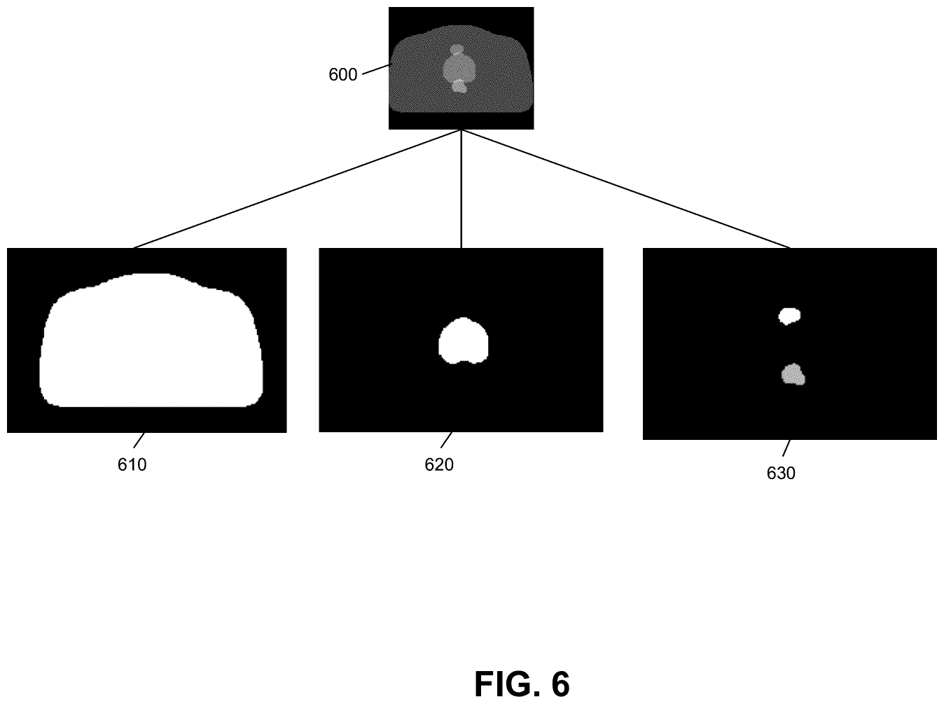

Referring to FIG. 6, a non-limiting example of a separation of a medical image into component parts is shown. In the depicted example, the medical image 600 can be separated into three medical images, where a medical image 610 illustrates the body of the patient, a medical image 620 illustrates the PTV, and a medical image 630 shows two different OARs near the PTV.

After separating the medical image, the analytics server may generate a weighted anatomy tensor. The weighted anatomy tensor may be a 4-dimensional tensor containing three channels where each channel is a 3D anatomy mask (e.g., separated medical image).

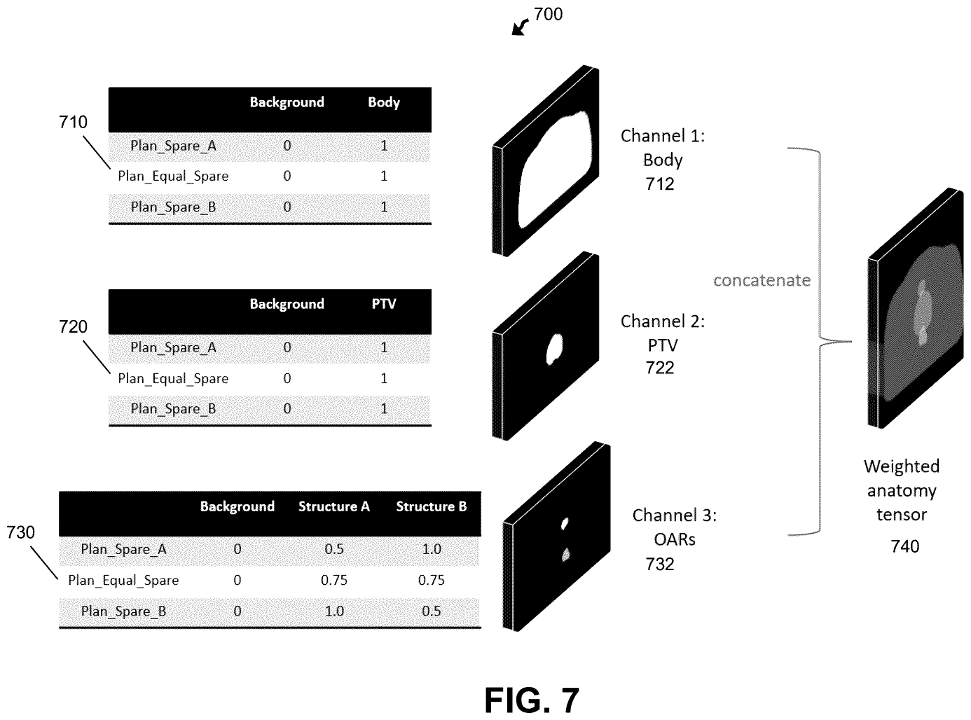

Referring now to FIG. 7, an example 700 illustrates how a weighted anatomy tensor can be generated. As depicted, a Channel 1 712 (corresponding to the medical image 610) may represent the body contour of a patient. As depicted in chart 710, the voxels that fall within the body may be assigned a value of 1 and those falling outside the body may be assigned a value of 0. This pattern may be true regardless of the type of the corresponding plans (Plan Spare A, Plan Equal Spare, or Plan Spare B).

A Channel 2 722 (corresponding to the medical image 620) may represent the PTV.

As depicted in chart 720, the voxels that fall within the PTV may be assigned a value of 1, and those falling outside the PTV may be assigned a value of 0. This pattern may be true regardless of the type of the corresponding plans (Plan Spare A, Plan Equal Spare, or Plan Spare B).

A Channel 3 732 (corresponding to the medical image 630) may represent the OARs, but the method of assigning values may differ from Channels 1 and 2, because the method may depend on the type of plan. As shown in the chart 730, for Plan Spare A, the voxels within Structure A may be assigned a value of 0.5, those within Structure B are assigned a value 1.0, and those in the background may be assigned a value of 0. For Plan Equal Spare, these values may be 0.75, 0.75, and 0 respectively. For Plan Spare B, the values may be 1.0, 0.5, and 0, respectively. The choice of these values can vary for other embodiments, as long as the values for Structures A and B add up to the same value, and a lower value for a structure indicates a lower dose to that structure. The analytics server may add different channels to generate the weighted anatomy tensor 740. That is, the weighted anatomy tensor 740 may be concatenated from the three depicted channels.

The analytics server may then train the AI model using the weighted tensors and the corresponding data (e.g., different plans). In some embodiments, the analytics server may use a conditional, variational auto-encoder (conditional VAE) to train the AI model. Accordingly, the analytics server may use VAEs or conditional VAEs to train the model. However, the analytics server may use other machine learning methods, as well.

The analytics server may encode the training dataset. For instance, the analytics server may use a VAE or conditional VAE to analyze the training data and compress various data within the training dataset, making it a latent space (e.g., a region in the latent space that represents similar data). In some examples, this can be referred to as “deconstruction” of the training dataset. In some examples, the model may use a dimensionality reduction protocol in which the content (within the training data) is transformed into a latent space. The latent space may refer to a mathematical representation of the weighted anatomy tensor (e.g., a mathematical space that maps back to the weighted anatomy tensor ingested by the AI model). By representing data in the latent space, the AI model may analyze the training dataset, as opposed to comparing them in their original form (e.g., medical images). Therefore, training the AI model using VAEs may create efficiencies not present in generically trained AI models.

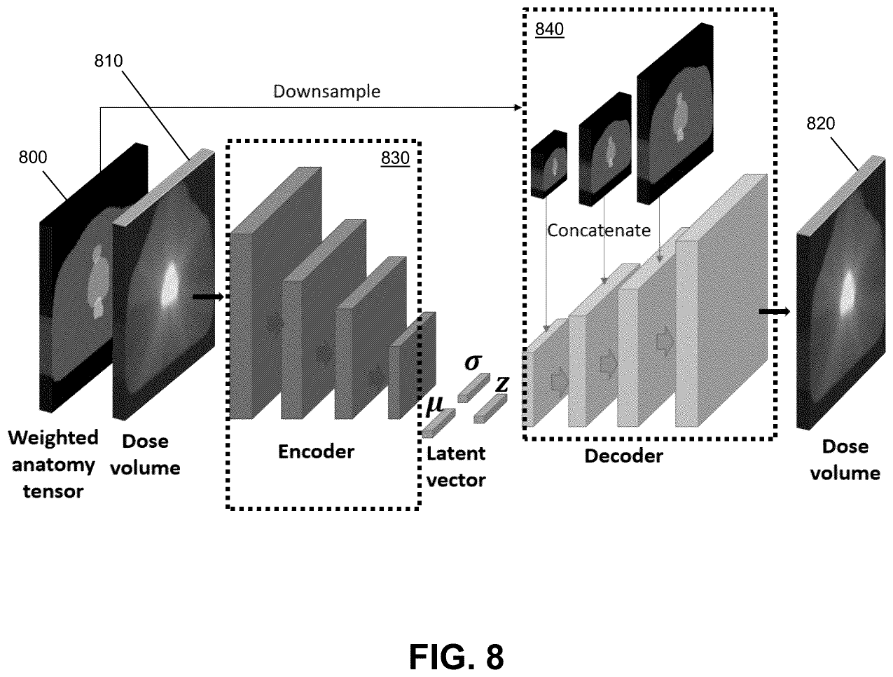

Referring now to FIG. 8, a non-limiting flow diagram of AI model training is depicted in accordance with an embodiment. To train the AI model, the weighted anatomy tensor 800 (e.g., the weighted anatomy tensor 740) and corresponding dose volume 810 are ingested by the AI model. The weighted anatomy tensor 800 and dose volumes 810 may be initially concatenated as a single tensor, which is converted by an encoder to the mean (u) and variance (a) of N Gaussian distributions. Subsequently, one value may be sampled from each of the N Gaussian distributions to form a latent vector of N elements. This vector, together with three downsampled weighted anatomy tensors, may be ingested by the decoder to output a reconstructed dose volume 820. During training, the AI model may encode the data using 3D convolutions (encoder 830) and then decode the data using upsampling and concatenating (840).

The loss function used for training may be an aggregate of reconstruction loss (between the original and the reconstructed dose volumes) and the Kullback-Leibler divergence loss (between the latent distribution and a Gaussian distribution centered at 0, with a variance of 1). Mathematically, the loss function can be expressed as:

$$z = latent vector$$

$$x = input data$$

$$Encoder = {q}_{θ} ( z \vert x )$$

$$Decoder = {p}_{φ} ( x \vert z )$$

$$p ( z ) = Normal ( 0 , 1 )$$

$$Loss = Reconstruction Loss + Regularizer$$

$$Loss = \text{?} [ log {p}_{φ} ( x \vert z ) ] - KL ( {q}_{θ} ( z \vert x ) ‖ p ( z ) )$$

$$\text{?} \text{indicates text missing or illegible when filed}$$

### Execution and Implementation of the AI Model

During training, the analytics server may iteratively produce new predicted results (recommendations) based on the training dataset. If the predicted results do not match the known or ground truth outcome, the analytics server continues the training unless and until the computer-generated recommendation satisfies one or more accuracy thresholds and is within acceptable ranges. For instance, the analytics server may segment the training dataset into three groups (i.e., training, validation, and testing). The analytics server may train the AI model based on the first group (training). The analytics server may then execute the (at least partially) trained AI model to predict results for the second group of data (validation). The analytics server then verifies whether the prediction is correct. Using the above-described method, the analytics server may evaluate whether the AI model is properly trained. The analytics server may continuously train and improve the AI model using this method. The analytics server may then gauge the AI model's accuracy (e.g., area under the curve, precision, and recall) using the remaining data points within the training dataset (test).

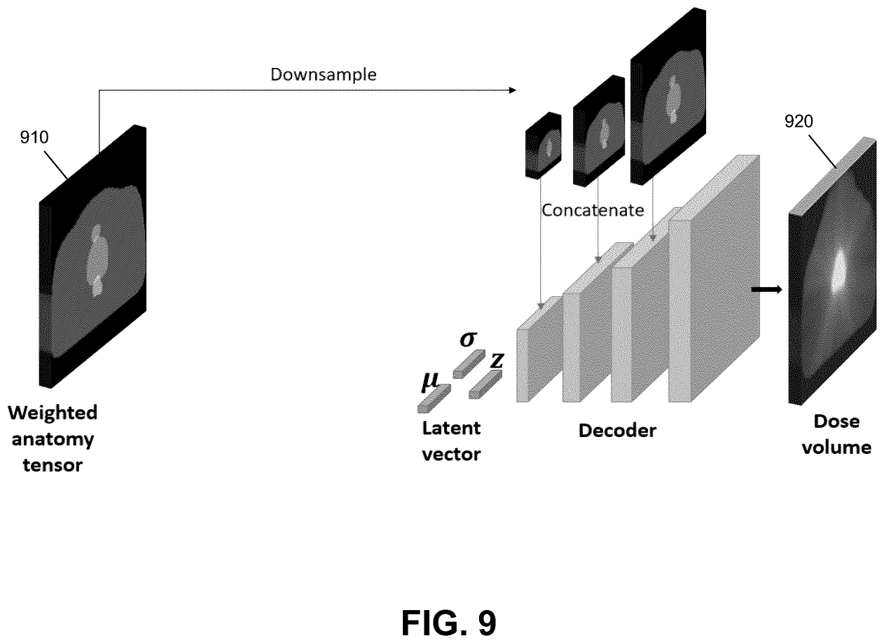

At implementation, the AI model may generate multiple prioritized dose distributions. Referring now to FIG. 9, a non-limiting flow diagram of AI model training is depicted in accordance with an embodiment. As depicted, the method 900 is partially similar to the training process depicted in FIG. 8. However, in the method 900, the VAE may not be used anymore, and the AI model may not ingest a dose volume.

Generating doses with varying degrees of trade-off patterns may be achieved by manipulating the values in the weighted anatomy tensor, as shown in Table 1:

TABLE 1

| | Background | Structure A | Structure B |
|---|---|---|---|
| Voxel value | 0 | w | 1.5 − w |

As illustrated, voxels that fall outside of any structures, within Structure A, or within Structure B may be assigned values of 0, w, and 1.5-w, respectively. The value w can be any float number between 0.5 and 1.0. In some embodiments, the closer w is to 0.5, a lesser dose will be delivered to Structure A and a greater dose will be delivered to Structure B.

To execute the trained AI model for dose generation, the input may include a weighted anatomy tensor 910 and a latent vector consisting of N elements, each sampled from a Gaussian distribution. As a result, the trained AI model predicts the dose volume 920.

Referring back to FIG. 2, at 210, the analytics server may receive a value indicating a prioritization between a first OAR of a patient and a second OAR of the patient receiving radiation dosage. The analytics server may present an electronic platform that includes one or more input elements configured to receive a value indicating whether (and how much) a clinician prefers to prioritize one OAR with respect to another OAR. For instance, the analytics server may display an input element that resembles a spectrum ranging from no prioritization to a full prioritization between the OARs. For instance, the spectrum (sliding scale that can be interacted with by the end-user) may include a range of 0-1 or 0-100 where 0 represents no prioritization (or a corresponding OAR) and 100 represents that the OAR is fully prioritized over the second OAR. In some embodiments, a different scale (e.g., 0-1) may be used. In another example, the analytics server may display an input element (e.g., radio button or a drop down menu) with which the clinician can input a numerical representation of how to prioritize one OAR with respect to another OAR.

At 204, the analytics server may execute an artificial intelligence model using the value to predict a radiation dosage for the first OAR, the second OAR, and a target structure, wherein the artificial intelligence model is trained in accordance with a training dataset comprising a set of participants, dosage administered to the participant's target structure, first OAR, and second OAR.

The analytics server may execute the AI model discussed herein using the data received in step 210. Additionally, the analytics server may receive data associated with the patient, such as treatment data, clinical objectives (e.g., dosage limits for PTV and/or OAR), or one or more medical images associated with the patient. Using the data received in step 210 and the additional data retrieved, the analytics server may execute the trained AI model. As a result, the AI model may predict different dose distribution values for the PTV and different OARs. In some embodiments, the trained AI model may generate DVHs predicting the dose distribution for the PTV and OARs.

At step 230, the analytics server may output the predicted radiation dosage for at least one of the first OAR, the second OAR, and a target structure.

The analytics server may output the predicted results of executing the AI model in multiple ways. In one embodiment, the analytics server may output the predicted results on a GUI. For instance, a GUI accessed by a medical professional may populate different dose distribution and their corresponding prioritization values. For instance, the GUI may show multiple DVHs and a graphical indicator illustrating which (if any) and how much an OAR was prioritized (spared).

In another example, the analytics server may transmit the data predicted by the AI model to a downstream application. For instance, the dose predictions may be transmitted to a plan optimizer where the predicted data (in conjunction with other treatment requirements and clinical objectives) can be used to generate a detailed treatment plan for the patient. Alternatively, the AI model discussed herein may be incorporated into and become a part of the plan optimizer itself.

In another example, the analytics server may revise one or more attributes of the patient's radiotherapy treatment using the data predicted by the AI model. For instance, the analytics server may revise an attribute of a multi-leaf collimator (MLC), move the couch, pause the beam, or a combination of any of these examples. Specifically, in conjunction with one or more other software solutions, the analytics server may revise an opening of the MLC, such that radiation dissemination is directed in accordance with the AI model's prioritized dose distribution.

### Non-Limiting Example

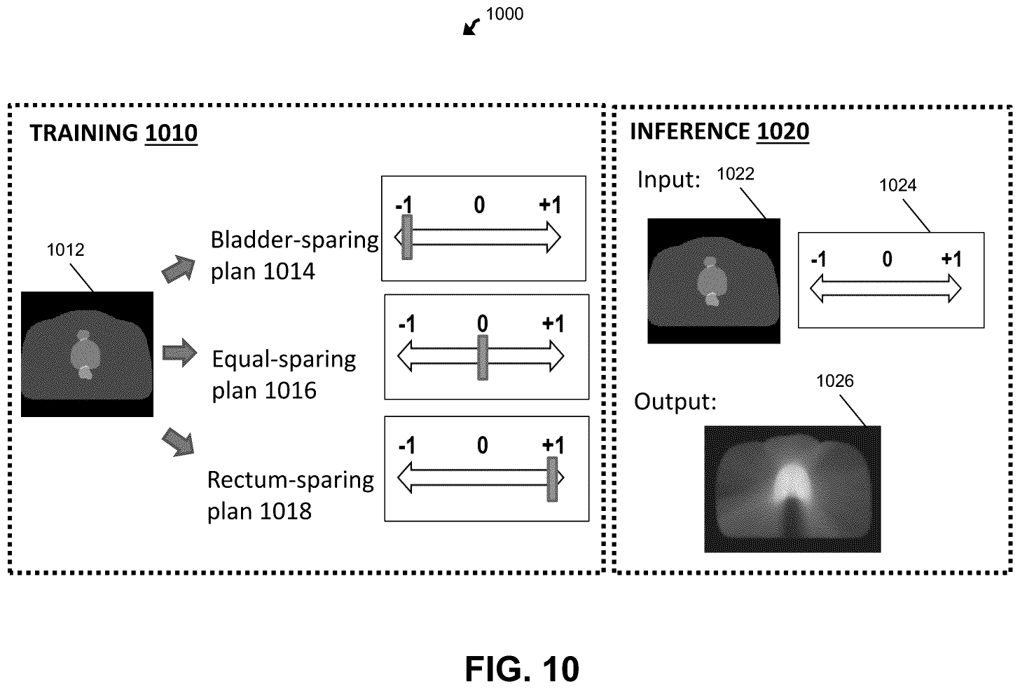

In a non-limiting example, the analytics server may train an AI model (using the methods discussed herein) to generate predicted dose distributions based on organ prioritization and tradeoffs. Referring now to FIG. 10, the method 1000 illustrates operational steps to train and execute an AI model to predict dose distributions for a patient being treated for prostate cancer. In the depicted embodiment, the patient's prostate is considered as the PTV, while their bladder and rectum are considered the OARs. Using the methods and systems discussed herein, the analytics server may generate dose distributions that prioritize (spare) one OAR in favor of the other while all organs remain within the clinical dosage limits and/or other clinical objectives required by a clinician.

As illustrated, in a training phase 1010, the analytics server may generate three plans including three different dose distributions for a medical image 1012. In the depicted embodiment, the analytics server may generate a first plan that prioritizes or spares the bladder (Bladder-Sparing Plan 1014), a second plan that treats both OARs equally (Equal-Sparing Plan 1016), and a third plan that prioritizes or spares the patient's rectum (Rectum-Sparing Plan 1018). Each plan can also include a numerical representation indicating how OARs are prioritized. For instance, each plan may include a numerical value (range of 0-1) that indicates its prioritization. For instance, the Bladder-Sparing Plan 1014 may be assigned a value of −1, which indicates that the plan prioritizes and spares the bladder over rectum. In contrast, the rectum-sparing plan is assigned a +1 value. Moreover, the equal-sparing plan may be assigned a 0 value to indicate its neutrality.

After training, the AI model may be configured to receive the value indicating the prioritization between the two OARs. Using the methods and systems discussed herein, the analytics server may train the AI model, such that at inference time, the trained AI model may receive a value indicating how the user desires to prioritize OARs and predicts various doses. For instance, during the inference phase 1020, the analytics server may retrieve the medical image 1022 (showing the patient's bladder, prostate, and rectum). The user may then input a value indicating their desired prioritization (e.g., using a sliding scale 1024). As a result, the AI model may output a dose distribution in accordance with the input (including the medical image 1026).

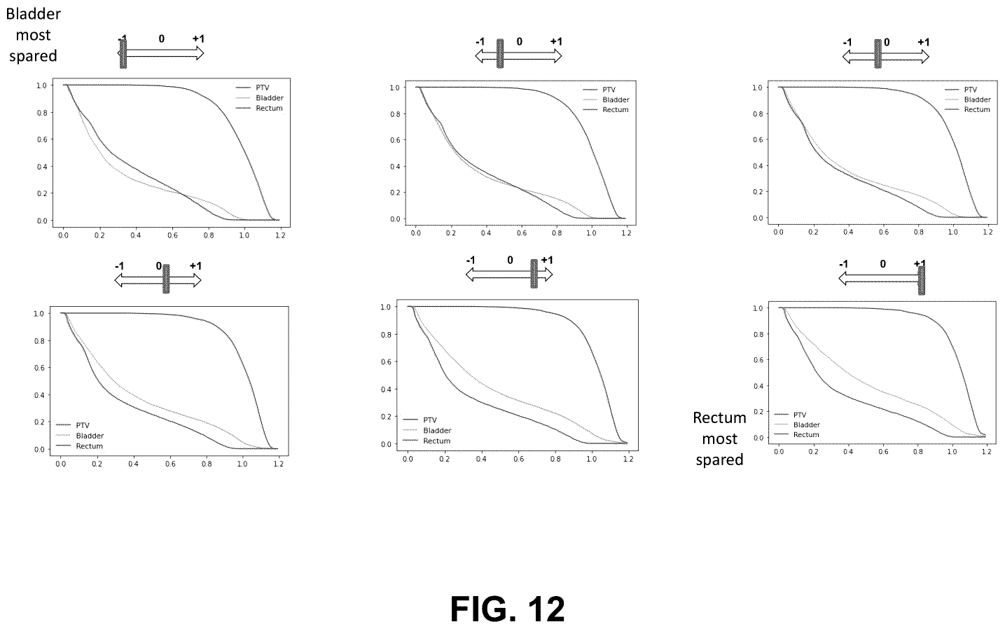

Even though the AI model is trained using three iterations of the same plan (plan 1014 having a value of −1, plan 1015 having a value of 0, and plan 1018 having a value +1), the AI model may generate a prediction for any input received that is within the same range. Therefore, the value received via the sliding scale 1024 is not limited to −1, 0, or +1. For instance, if the clinician inputs a value of 0.35 (using the sliding scale 1024 or otherwise), the AI model may accordingly interpolate a prioritized prediction of dose distributions. Different DVHs corresponding to different prioritization values are depicted in FIG. 12.

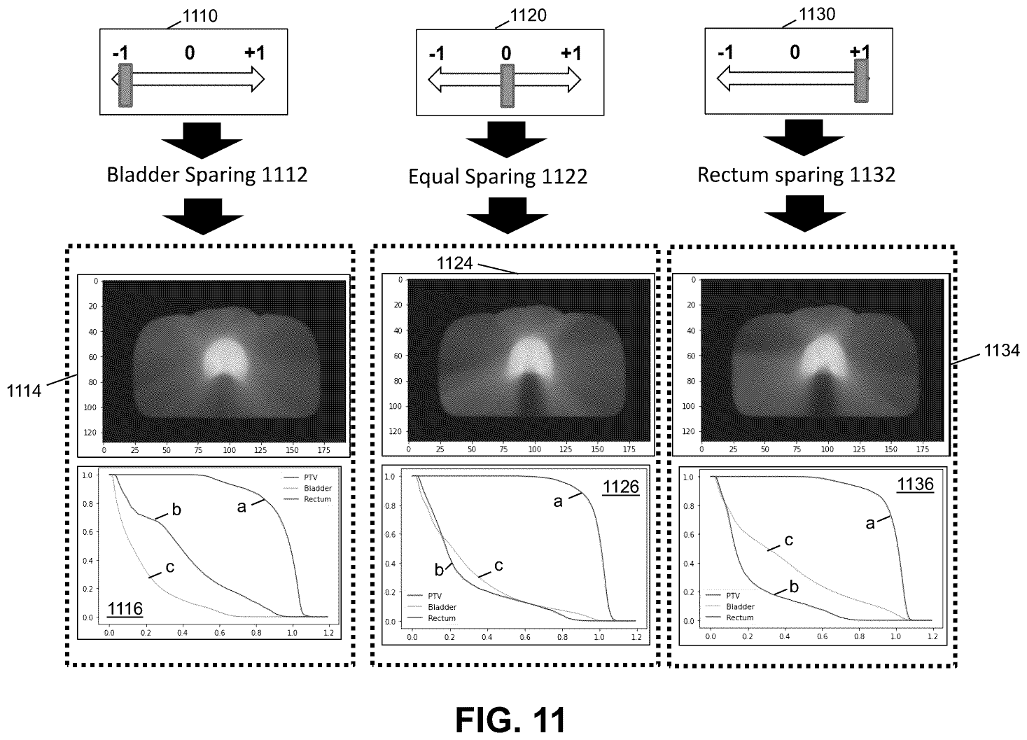

In another non-limiting example depicted in FIG. 11, a clinician accesses a software application that is hosted (or otherwise related to the analytics server). Using a sliding scale, the clinician may indicate a value associated with how the clinician would like to prioritize OARs receiving dosage during treatment. Depending on this input, the analytics server may predict different dosages. For instance, if the clinician selects −1 via the sliding scale 1120, the analytics server may generate the Bladder Sparing Plan 1112, if the clinician selects 0 via the sliding scale 1120, the analytics server may generate the Equal Sparing Plan 1122, if the clinician selects +1 via the sliding scale 1120, the analytics server may generate the Rectum Sparing Plan 1132.

Each plan may include a medical image (medical images 1114-1124, and 1132). Each plan may also include a DVH that corresponds to the dosage values predicted by the AI model. For instance, the Bladder Sparing Plan 1110 may include the DVH 1116 that includes a line a (corresponding to dosage values of PTV), line b (corresponding to dosage values of the rectum), and line c (corresponding to dosage values of the bladder). As depicted, the dosage value for the bladder (line c) is lower than the dosage values of the rectum (line b).

The Equal Sparing Plan 1120 may include the DVH 1126 that includes lines a (corresponding to dosage values of PTV), b (corresponding to dosage values of the rectum), and c (corresponding to dosage values of the bladder). As depicted the dosage value for the bladder is similar (within a tolerable threshold) to the value of a dosage value of the rectum. In some embodiments, the dosage value of the bladder and rectum will not be similar to each other. However, the dosage value for each organ will not be prioritized or spared when compared to the Bladder Sparing Plan 1112 or the Bladder Sparing Plan 1110. Similarly, the Equal Sparing Plan 1122 generates a DVH 1126 that depicts dosage predictions that are substantially the same for the bladder (line b) and the rectum (line c).

For instance, the rectum sparing plan 1130 may include the DVH 1136 that includes line a (corresponding to dosage values of PTV), line b (corresponding to dosage values of rectum), and line c (corresponding to dosage values of the bladder). As depicted, the dosage value for the rectum (line b) is lower than the dosage values of the bladder (line c).

The clinician may revise the predicted dosages by interacting with the sliding scale, thereby revising the predicted values as many times as necessary to identify their desired plan (including the dosages that satisfy one or more criteria). Once the right plan is identified, the clinician may accept the plan where the plan is transmitted to a downstream software application and implemented for the patient's treatment.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What we claim is:

1. A method comprising:
receiving, by a processor, a value indicating a prioritization between a first organ at risk of a patient and a second organ at risk of the patient receiving radiation dosage;
executing, by the processor, an artificial intelligence model using the value to predict a radiation dosage for the first organ at risk, the second organ at risk, and a target structure, wherein the artificial intelligence model is trained in accordance with a training dataset comprising a set of participants, dosage administered to the participant's target structure, first organ at risk, and second organ at risk; and
outputting, by the processor, the predicted radiation dosage for at least one of the first organ at risk, the second organ at risk, or a target structure.

2. The method of claim 1, wherein outputting the predicted dosage comprises displaying a dose-volume histogram depicting the predicted radiation dosage for at least one of the first organ at risk, the second organ at risk, or a target structure.

3. The method of claim 1, wherein the value indicating the prioritization between the first organ at risk of the patient and the second organ at risk of the patient is received via a sliding scale input element.

4. The method of claim 1, wherein the processor receives a plurality of values indicating a plurality of prioritizations between the first organ at risk of the patient and the second organ at risk of the patient receiving radiation dosage and outputs a plurality of predicted radiation dosages.

5. The method of claim 1, further comprising:
transmitting, by the processor, the predicted radiation dosage to a plan optimizer software solution.

6. The method of claim 1, further comprising:
adjusting, by the processor, at least one attribute of a radiotherapy machine in accordance with the predicted radiation dosage.

7. The method of claim 1, wherein the artificial intelligence model is trained using a set of weighted tensors corresponding to the training dataset.

8. The method of claim 7, wherein the artificial intelligence model is trained using a generative artificial intelligence model corresponding to a variational auto-encoder or a conditional variational auto-encoder.

9. A computer system:
a server comprising a processor and a non-transitory computer-readable medium containing instructions that when executed by the processor causes the processor to:
receive a value indicating a prioritization between a first organ at risk of a patient and a second organ at risk of the patient receiving radiation dosage;
execute an artificial intelligence model using the value to predict a radiation dosage for the first organ at risk, the second organ at risk, and a target structure, wherein the artificial intelligence model is trained in accordance with a training dataset comprising a set of participants, dosage administered to the participant's target structure, first organ at risk, and second organ at risk; and
output the predicted radiation dosage for at least one of the first organ at risk, the second organ at risk, or a target structure.

10. The system of claim 9, wherein outputting the predicted dosage comprises displaying a dose-volume histogram depicting the predicted radiation dosage for at least one of the first organ at risk, the second organ at risk, or a target structure.

11. The system of claim 9, wherein the value indicating the prioritization between the first organ at risk of the patient and the second organ at risk of the patient is received via a sliding scale input element.

12. The system of claim 9, wherein the processor receives a plurality of values indicating a plurality of prioritizations between the first organ at risk of the patient and the second organ at risk of the patient receiving radiation dosage and outputs a plurality of predicted radiation dosages.

13. The system of claim 9, wherein the instructions further cause the processor to transmit the predicted radiation dosage to a plan optimizer software solution.

14. The system of claim 9, wherein the instructions further cause the processor to adjust at least one attribute of a radiotherapy machine in accordance with the predicted radiation dosage.

15. The system of claim 9, wherein the artificial intelligence model is trained using a set of weighted tensors corresponding to the training dataset.

16. The system of claim 15, wherein the artificial intelligence model is trained using a generative artificial intelligence model corresponding to a variational auto-encoder or a conditional variational auto-encoder.

17. A system comprising:
a computer in communication with a server and configured to display a graphical user interface;
a radiotherapy machine in communication with the server; and
the server configured to:
receive a value indicating a prioritization between a first organ at risk of a patient and a second organ at risk of the patient receiving radiation dosage;
execute an artificial intelligence model using the value to predict a radiation dosage for the first organ at risk, the second organ at risk, and a target structure, wherein the artificial intelligence model is trained in accordance with a training dataset comprising a set of participants, dosage administered to the participant's target structure, first organ at risk, and second organ at risk; and
output the predicted radiation dosage for at least one of the first organ at risk, the second organ at risk, or a target structure.

18. The system of claim 17, wherein outputting the predicted dosage comprises displaying a dose-volume histogram depicting the predicted radiation dosage for at least one of the first organ at risk, the second organ at risk, or a target structure.

19. The system of claim 17, wherein the value indicating the prioritization is received via a sliding scale input element.

20. The system of claim 17, wherein the server receives a plurality of values indicating a plurality of prioritizations between the first organ at risk of the patient and the second organ at risk of the patient receiving radiation dosage and outputs a plurality of predicted radiation dosages.
